(19) United States
(12) Patent Application Publication
(10) Pub. No.: US 2024/0094179 A1
(43) Pub. Date: Mar. 21, 2024

(54) METAL OXIDE-POLYMER NANO COMPOSITE FOR AMMONIA SENSING AT TEMPERATURES BELOW AMBIENT INCLUDING SUB-ZERO TEMPERATURES

(71) Applicant: COUNCIL OF SCIENTIFIC AND INDUSTRIAL RESEARCH, New Delhi (IN)

(72) Inventors: Swastik MONDAL, Kolkata (IN); Nirman CHAKRABORTY, Kolkata (IN)

(21) Appl. No.: 18/264,238

(22) PCT Filed: Mar. 26, 2022
(86) PCT No.: PCT/IN2022/050309

§ 371 Date: Aug. 3, 2023

(30) Foreign Application Priority Data
Mar. 31, 2021 (IN) 202111015506

(51) Int. Cl. G01N 33/00

(52) U.S. Cl. CPC G01N 33/0054

(57) ABSTRACT

Antimony doped tin oxide-polypyrrole nano-composites for in situ and ex situ ppm level ammonia gas sensing at very low temperatures (room temperature to sub-zero ° C.). During synthesis, hydrogen peroxide (H₂O₂) has been used as a novel template for simultaneous oxidative polymerization of pyrrole as well as the polymer loading on doped metal oxide base. Usability of the above composition was tested for ppm level in-situ and ex-situ ammonia gas sensing in a very low temperature range (room temperature to −30° C.). Stable and highly selective responses to ammonia gas were recorded in situ and ex situ at the very low temperature range (room temperature to −30° C.). The revelation of very low temperature ammonia sensing property by the antimony doped tin oxide-polypyrrole nano-composite could be used for detecting meat and meat product spoilage under in situ and ex situ refrigerated conditions for cold storages and commercial preservations.

The present invention relates to a novel metal oxide-polymer nano composite for very low temperature (below room temperature to −30° C.) sensing of low ppm ammonia gas.

### FIELD OF THE INVENTION

The present invention relates to a novel metal oxide-polymer nano composite for very low temperature (below room temperature to −30° C.) stable and selective ex-situ & in-situ ammonia gas detection. In the synthesis process, hydrogen peroxide (H₂O₂) has been used for the first time, simultaneously, for in-situ oxidative polymerization of pyrrole as well as for its loading on the doped metal oxide system.

### BACKGROUND OF THE INVENTION

Ammonia gas ranging from low to high ppm concentration is a major gaseous component for monitoring environmental pollution caused due to industries, chemical companies, waste degradation, and vehicular pollution as well as for detecting physical ailments as health bio-markers and also as an indicator of meat and meat product spoilage. In today's world of industrialization, packed and frozen meat/meat products has become an inevitable part of the huge consumer society where annually several tons of packed and frozen meat/meat products become a part of a country's economy, both in terms of domestic production and import & export. India has been proudly exporting meat (under packed and frozen conditions) but there are reports of annual spoilage of about 2.71% of meat and 6.74% of poultry meat due to improper cold storage conditions. While India is the second largest exporter of meat, it is also globally the largest producer of buffalo meat and second largest producer of goat meat. And on a global scale, despite refrigeration chains, chemical preservatives and recent methods, around 25% of such meat generated post harvest or slaughter is altered due to microbial spoilage and becomes unfit for consumption.

Hence, for economies which depend a lot on trades in packed, frozen meat; in-situ and ex-situ detection of meat spoilage under frozen conditions is an efficient and invincible tool for early identification of meat deterioration and undertaking necessary steps to prevent further spoilage.

Ammonia gas is one of the major volatile amines that are released due to spoilage of meat or meat products. Accordingly, regular checking of presence of trace ammonia in stored meat products under refrigerated conditions shall ensure their quality and edibility standards.

Gas and vapor sensors have been successfully used in detecting and monitoring ammonia gas in a wide range of concentrations (ppb to 1000 ppm) and at different temperatures ranges, wherein, the lower limit being room temperature. Tailoring pure and doped metal oxide systems with polymers and surface additives have successfully brought down the sensor operation at room temperature. But besides ammonia pollution and leakage at room temperature and high temperatures, commercial detection of trace ammonia at temperatures below room temperature to sub-zero temperature is an inevitable sphere where further sensor development is needed. This puts forward the need of selective and stable low temperature ammonia sensing.

Reference may be made to Wang Y., Mu Q., Wang G., Zhou Z., Sensors and Actuators B: Chemical, 145 (2), 847-853 (2010) “Sensing characterization to NH₃ of nanocrystalline Sb-doped SnO₂ synthesized by a nonaqueous sol-gel route” wherein antimony doped tin oxide nanoparticles were synthesized using inorganic precursors like SnCl₄, Sb(OC₂H₅)₃) and the cationic surfactant (cetyltrimethylammonium bromide, CTAB). As compared to pure tin oxide, the ATO nano particles showed a 2.5 times improved ammonia sensing response at a temperature of 79° C. The drawback of the work is its limitation of working temperature to 79° C.

Reference may be made to Joulazadeh M., Navarchian A. H., Synthetic Metals Volume 210, Part B, 404-411 (2015) “Ammonia detection of one-dimensional nano-structured polypyrrole/metal oxide nanocomposites, sensors” wherein polypyrrole loaded tin oxide/zinc oxide nano-composites were used to detect various ppm of ammonia at room temperature. The drawback of the work is its limitation of working temperature to room temperature.

Reference may be made to Seekaew Y., Pon-On W., Wongchoosuk C., ACS Omega 4(16), 16916-16924(2019) “Ultrahigh Selective Room-Temperature Ammonia Gas Sensor Based on Tin-Titanium Dioxide/reduced Graphene/Carbon Nanotube Nanocomposites by the Solvothermal Method” wherein ultrahigh selective NH₃ gas sensor based on tin-titanium dioxide/reduced graphene/carbon nanotube (Sn—TiO₂@rGO/CNT) nanocomposites were synthesized. The Sn—TiO₂@rGO/CNT nanocomposite gas sensor with molar ratio of Sn/Ti=1:10 showed the highest response to NH₃ over other molar ratios of Sn/Ti as well as pure rGO/CNT and Sn—TiO₂ gas sensors at room temperature. The drawback of the work is its limitation of working temperature to room temperature and use of complex chemical compositions.

Reference may be made to Chinese patent no. CN105136869B by Li Y., Ban H., Yang M., “Polyaniline/nano-iron oxide composite resistance type material sensor and preparation method thereof” wherein a polyaniline/nano-iron oxide composite was used for detecting low concentration ammonia (<100 ppm) effectively at room temperature. The drawback of the work is its limitation of working temperature to room temperature

Reference may be made to Chinese patent no. CN109580739A by Huiling T., Chunhua L., Hong P., Junxin Z., Yadong J., Zhen Y., Guangzhong X., Xiaosong, D. wherein a flexible ammonia sensitive film is made of conductive organic polymer material or metal oxide doped conductive organic polymer composite material or conductive organic polymer composite materials doped with carbon-based materials or conductive organic polymer composite materials doped with graphene-like materials for low ppm ammonia sensing at room temperature. With a low ppm ammonia concentration range (0.2 to 10 ppm), a higher sensitivity and better resistance to moisture at low temperature has been achieved. The drawback of the work is its limitation of working temperature to room temperature or below the room temperature.

The drawbacks of the above known ammonia sensing systems are discussed herein. The material compositions as disclosed and used in these prior art documents have their standard operating temperatures limited till room temperature. Accordingly, detection of ammonia at temperatures below room temperature to sub-zero ° C. hasn't been achieved.

Further, very low temperature (below room temperature to sub-zero ° C.) ex-situ and in-situ sensing of gas/VOCs hasn't been observed.

Furthermore, use of a sole material/chemical for oxidative polymerization (of monomer) as well as loading of the polymer on the metal oxide base matrix hasn't been exhibited.

### Objectives of the Invention

The main objective of the present invention is to provide a novel metal oxide-polymer nano composite for very low temperature (below room temperature to sub-zero ° C.) sensing of low ppm ammonia gas which obviates the drawbacks of the hitherto known prior art as detailed above.

Another objective of the present invention is to investigate the utility of novel (2-9 atomic %) antimony doped tin oxide-poly pyrrole nano-composite for detecting ammonia in very low temperature (below room temperature to −30° C.) environment.

Still another objective of the present invention is to achieve selective detection of ammonia gas at very low temperature range using a novel metal oxide-polymer nano-composite.

Yet another objective of the present invention is to explore the utility of hydrogen peroxide (H₂O₂) in simultaneous in-situ oxidative polymerization of pyrrole as well as the loading of poly-pyrrole on antimony doped tin oxide.

Still another objective of the present invention is to provide a composition that can detect low ppm ammonia gas under ex-situ and in-situ very low temperature conditions (below room temperature to −30° C.) by antimony doped tin oxide-poly pyrrole nano composite.

### SUMMARY OF THE INVENTION

The present invention provides antimony doped tin oxide-polymer nano composites for low ppm ammonia gas sensing under ex-situ and in-situ very low temperature (below room temperature to −30° C.) conditions. For low concentration ammonia sensing (5-100 ppm) at 4° C., a swift response in the range of 40%-80% was observed, which was also highly selective in presence of humidity and a range of gas/VOCs. Further, low ppm ammonia gas sensing response is recorded for sub-zero temperature range, for example, at around −2° C. the ammonia gas sensing response is around 50%-88% and at −30° C., a stable response in range of 60%-80% was recorded.

Further, the present invention provides a method for synthesis of antimony doped tin oxide-polymer nano composites. During synthesis of the antimony doped tin oxide-polymer nano composites, hydrogen peroxide (H₂O₂) has been used as a novel agent for oxidative polymerization of pyrrole as well as the loading of antimony doped tin oxide matrix by poly pyrrole. The very low temperature sensing capability of the said nano composites could be used for ex-situ and in-situ detection of spoiled meat/meat products stored under refrigerated conditions.

Furthermore, the present invention provides method for detection of spoiled meat/meat products stored under refrigerated conditions.

### BRIEF DESCRIPTION OF THE DRAWING

To further clarify advantages and aspects of the present antimony doped tin oxide-polypyrrole nano-composite, a more particular description of the present nano-composite will be rendered by reference to specific embodiments thereof, which is illustrated in the appended drawing(s). It is appreciated that the drawing(s) presented herein depicts only typical embodiments of the invention and are therefore not to be considered limiting of its scope.

FIG. 1: represents highly selective 30 ppm ammonia gas (both in air and N2 medium) sensing at 4° C. and N1S spectrum of the loaded conducting polymer (polypyrrole); and

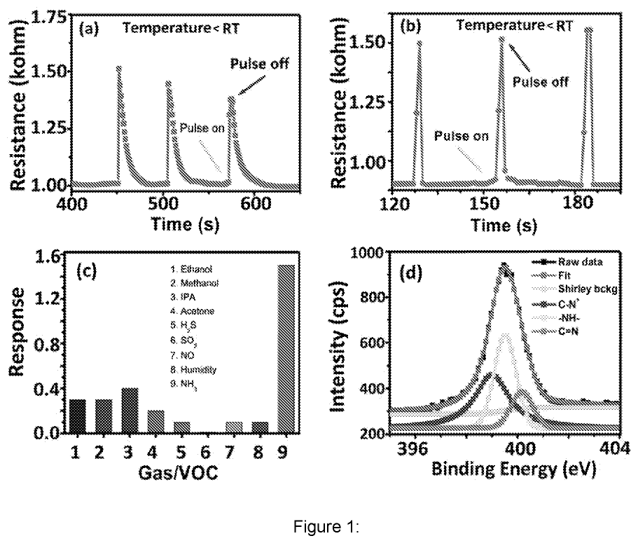

FIG. 2: represents very low temperature ammonia sensing set-up with substrate and material coating used.

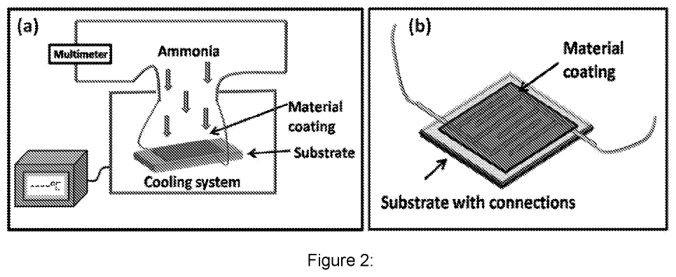

### DETAILED DESCRIPTION OF THE INVENTION

Accordingly the present invention provides a composition for very low temperature (below room temperature to sub-zero ° C.) sensing of low ppm ammonia gas, wherein, the said composition consist of 2-9 atomic % antimony doped tin oxide-polypyrrole nano-composite.

In another embodiment the antimony doped tin oxide-polypyrrole nano-composite includes 1-2.5 wt. % of polypyrrole. Further, a particle size of the antimony doped tin oxide is in a nano particle size range (10 to 30 nm), wherein, the said nano particle size increases a surface to volume ratio and thus improves overall sensing response. Further, the said composition shows a stable base resistance at temperature below room temperature to −30° C.

Further, the present invention also provides a method for preparing a composition for very low temperature sensing of low concentration ammonia gas, wherein, the method comprises steps of polymer loading, which uses hydrogen peroxide (H₂O₂) for simultaneous oxidative polymerization of pyrrole. Further, the method includes loading of in-situ synthesized polypyrrole on the antimony (2-9 atomic %) doped tin oxide matrix.

In another embodiment, the composition of the present invention provides a selective, stable and reproducible response to low ppm (5-100 ppm) ammonia gas at low temperature (below room temperature to −30° C.).

In one embodiment of the present invention, antimony has been incorporated in tin oxide as dopant.

In another embodiment of the present invention, the antimony doped tin oxide has been synthesized by the following steps:
(a) preparing a clear solution of SnCl₄·5H₂O in distilled water followed by magnetic stirring and also preparing a clear solution of SbCl₃·2H₂O in distilled water.
(b) mixing of the two separate salt solutions as prepared in step (a) together under prolonged magnetic stirring for several hours followed by addition of ammonia drop-wise till a pH of 9 was obtained.
(c) the gel type precipitate formed was kept under prolonged magnetic stirring and centrifuged in ethanol medium to get the clean gel.
(d) the gel was then dried and calcined at 800-1000° C. for obtaining the powder samples.

In another embodiment of the present invention, pyrrole solution (1 M-2.5 M in water) is polymerized in-situ while loading on the said antimony doped tin oxide using hydrogen peroxide (H₂O₂) for simultaneous oxidative polymerization as well as polymer loading on tin oxide matrix.

In another embodiment of the present invention, the obtained material was drop-casted on a flat sensor and allowed to dry at 50-100° C. for 6-7 hours and then placed in a freezer to record the base resistance.

In yet another embodiment of the present invention, stable responses to low ppm ammonia (5 to 100 ppm) have been exhibited by the obtained materials when used as a sensor under very low temperature conditions (i.e., below room temperature to −30° C.).

In still another embodiment of the present invention, cross sensitivity tests under very low temperature conditions (below room temperature to −30° C.) with several gas/VOCs including humidity have been performed.

Yet another embodiment of the present invention provides a chemically and physically stable composition for very low temperature (below room temperature to −30° C.) sensing of low ppm ammonia gas which comprises of poly-pyrrole loaded antimony doped tin oxide.

Hence, the present invention provides a novel metal oxide-polymer nano-composite for very low temperature (below room temperature to −30° C.) in-situ and ex-situ sensing of low ppm ammonia gas. While the response is reproducible and stable at the low temperature regime, it is highly selective to ammonia as compared to a wide range of gas/VOCs including humidity. Specifically, the material used for ammonia sensing is poly-pyrrole loaded antimony doped tin oxide nano-composite, wherein hydrogen peroxide has been used as an oxidizing agent for simultaneous oxidative polymerization of pyrrole monomer and loading of in-situ synthesized polymer on antimony doped tin oxide. The steady state operational temperature range of antimony doped tin oxide is thus brought below room temperature till sub-zero ° C.

### Gas and Vapor Sensing Experiments:

Gas and vapor sensing experiments have categorically been limited to room temperature as a lower limit with the upper limit extending to several hundred degrees. Particularly with ammonia, the lowest temperature to which it has been selectively sensed is room temperature. For metal oxides (pure and doped), bringing down the operational temperature below that of room has been a major challenge. Even with loading and intercalation with several conducting polymers, this has been a stumbling block in the spectrum of gas/vapor sensing.

However, in the present invention, the antimony doped tin oxide is loaded with poly-pyrrole and have exposed to low concentration (5-100 ppm) ammonia pulses at different low temperature and below room temperature to sub-zero temperature. Application of low ppm ammonia pulses at 4° C. leads to a hike in sample resistance, the response for 30 ppm ammonia being around 40%-80%. The response time for each pulse is only a few seconds and is reproducible and highly selective. For sub-zero range, response recorded, for example, at around −2° C. is around 50%-88% and at −30° C., a stable response in range of 60%-80% was recorded.

The different steps of the low temperature ammonia sensing experiment are as follows:
i) In the first step, antimony doped tin oxide nanoparticles of formula Sn₁₋ₓSbₓO₂ were synthesized by a co-precipitation method followed by prolonged magnetic stirring. Measured amount of tin and antimony solutions were separately dissolved in D.I. water and stirred for several hours. The clear solutions were then mixed, followed by adding ammonia solution (25%) drop-wise till a pH of around 9-10 was obtained. The precipitate was allowed to undergo continuous stirring for several hours followed by centrifugation and drying. The as prepared sample was then calcined at a high temperature of 800-1000° C., followed by phase analysis using XRD.
ii) In next step, measured amount of hydrogen peroxide (H₂O₂) was added to a small amount of the antimony doped tin oxide sample followed by drop-wise adding of water. The mixture was then kept under magnetic stirring for few hours. Pyrrole solution was added separately in distilled water and then poured in the tin oxide mixture. The whole solution was now allowed to undergo heat treatment at 100-150° C. for several hours. The sample was finally washed several times by ethanol and dried.
iii) The next step is to fabricate the sensor. For this, a small amount of powder sample (0.1 g) formed was taken in a mortar pestle and made into a consistent paste in iso-propyl alcohol. The slurry was then taken by a micropipette and drop casted on a flat substrate followed by drying at 60° C. The connections were made by platinum wire with contacts made with silver paste. The sensor module was placed in the freezer where temperature could be varied below room temperature to −30° C. The connections with multi-meter were drawn out from the freezer for resistance measurements. The multi-meter in turn was attached to a computer using IR cable for recording the dynamic responses at different low temperature slabs.
iv) Before the sensing experiments were performed, the sensing set-up was calibrated at different temperatures below room temperature till −30° C.
v) For sensing experiments, ammonia gas in different concentration (5-100 ppm) was prepared in air by desiccator dilution method. Gas pulses of different concentration were applied on the sensor head kept inside the freezer using a syringe and rubber pipe. For ammonia gas in nitrogen mixture, the gas inlet to the freezer was connected to the outlet of a MFC (Mass Flow Controller) and pulses of different concentration were separately applied to the sensor head under similar flow rate conditions. For 30 ppm ammonia gas at 4° C., maximum stable response in the range of 40%-80% was observed, with a swift response time of few seconds. Measurement at sub-zero range, for example, at −2° C. with 30 ppm ammonia gave a stable response in range of 50%-88% and at −30° C., a stable response in range of 60%-80%. In order to ensure that the response recorded by the system was purely due to ammonia and not due to air/nitrogen (used as a dilution medium for high concentration ammonia), blank experiments with air/nitrogen were also performed at the particular temperatures. The sample showed no responses to pure air/nitrogen, confirming the sensor response solely due to ammonia gas.
vi) Cross-sensitivity tests with humidity and several other gas(s)/VOC(s) were performed and it was observed that the system gives multifold times more response than humidity and all tested gas(s)/VOC(s) and is also highly sensitive to ammonia in particular.
vii) The poly-pyrrole loading by H₂O₂ has been identified by deconvoluting N1S peak obtained in the XPS spectrum of the metal oxide-polymer nano-composite. The amount of antimony doped into SnO₂ was analyzed by TEM-EDX (Transmission Electron Microscopy-Energy Dispersive X-ray) methods and ICP (Inductively Coupled Plasma) spectroscopic techniques.
viii) The chemistry behind the polymer loading and low temperature sensing could be explained as follows: antimony doped tin oxide is an n-type doped semiconductor whereas ammonia gas usually has a reducing nature. The in-situ oxidative polymerization and consecutive loading on the metal oxide base leads to generation of over-oxidized polypyrrole, which now bears positive holes on the carbon atoms of the pyrrole rings. This oxidative polymerization mediated hole generation makes the composite show a hike in resistance when exposed to a reducing gas like ammonia. Since poly-pyrrole is a conducting polymer and at lower temperature (below room temperature), holes have greater mobility than electrons, the over-oxidized poly-pyrrole loading enables hole mediated sensing which is preferable at low temperature regime.

The following examples are given by way of illustration of the working of the invention in actual practice and therefore should not be construed to limit the scope of the present invention in any way.

### EXAMPLES

### Example-1

Measured amount of H₂O₂ (5-10 ml) was added to 2-5 g of antimony doped tin oxide followed by drop-wise addition of water. The solution was allowed to undergo mixing for 2-4 hours to get a mixture. Then, 2-4 ml of aniline solution was added to 20 ml of D.I. water and then added to the mixture as prepared above. The obtained solution was then allowed to undergo prolonged heating at 120° C. for 5 hours. The solution was then centrifuged and washed with ethanol, dried at 80° C. for 3 hours and then drop-casted on the flat substrate.

The sensor was cured at 60° C. for 7 hours and connected to a multimeter for resistance measurements. The sensor was placed in a freezer. After a period of nearly 15-30 minutes, the sensor exhibited a stable base resistance in range of 1 W. This was checked for several hours to record any further fluctuations in the value. The sensor was then exposed to ammonia gas (air mixture) in range of 100-50 ppm, using a glass syringe and connecting silicon pipe.

Changes in resistance were observed with each pulse, where, a swift response time of few seconds was observed followed by a prolonged recovery. The ammonia pulses were repeated twice under similar conditions and the similarity in response was recorded.

Eventually, tests were done for lower ppm concentration and results were analyzed. Similar experiments were also performed with ammonia gas in nitrogen medium and changes following same pattern were observed.

During the experiments, the temperature of the freezer was varied from below room temperature to sub-zero degrees, changes in presence of ammonia individually recorded at intervals of 2° C.

### Example-2

Measured amount of H₂O₂ (5-10 ml) was added to 2-5 g of antimony doped tin oxide followed by drop-wise addition of water. The solution was allowed to undergo mixing for 2-4 hours to get a mixture. Then, 2-4 ml of carbazole solution was added to 20 ml of D.I. water and then added to the mixture as prepared above. The obtained solution was then allowed to undergo prolonged heating at 120° C. for 5 hours. The solution was then centrifuged and washed with ethanol, dried at 80° C. for 3 hours and then drop-casted on the flat substrate.

The sensor was cured at 60° C. for 7 hours and connected to a multimeter for resistance measurements. The sensor was placed in a freezer. After a period of nearly 15-30 minutes, the sensor exhibited a stable base resistance in range of 1 W. This was checked for several hours to record any further fluctuations in the value. The sensor was then exposed to ammonia gas (air mixture) in range of 100-50 ppm, using a glass syringe and connecting silicon pipe.

Changes in resistance were observed with each pulse, where, a swift response time of few seconds was observed followed by a prolonged recovery. The ammonia pulses were repeated twice under similar conditions and the similarity in response was recorded.

Eventually, tests were done for lower ppm concentration and results were analyzed. Similar experiments were also performed with ammonia gas in nitrogen medium and changes following same pattern were observed.

### Example-3

Measured amount of MWCNT (Multi-Walled CNT) was added to the solution of antimony and tin and precipitated simultaneously with ammonia. The solution was stirred for long 20 hours and then cleaned using ethanol solution. The material was then dried at 100° C. for 4 hours followed by calcination at 1000-1200° C. The MWCNT loaded antimony doped tin oxide powder was then coated on the flat substrate by drop casting method and cured at 60° C. for 7 hours. The sensor was then placed in the freezer and exposed to ammonia gas in range of 5 to 100 ppm.

### Example-4

Measured amount of H₂O₂ (5-10 ml) was added to 2-5 g of pure tin oxide followed by drop-wise addition of water. The solution was allowed to undergo mixing for 2-4 hours to get a mixture. Then, 2-4 ml of pyrrole solution was added to 20 ml of D.I. water and then added to the mixture as prepared above. The obtained solution was then allowed to undergo prolonged heating at 120° C. for 5 hours. The solution was then centrifuged and washed with ethanol, dried at 80° C. for 3 hours and then drop-casted on the flat substrate.

The sensor was cured at 60° C. for 7 hours and connected to a multimeter for resistance measurements. The sensor was placed in a freezer. After a period of nearly 15-30 minutes, the sensor exhibited a stable base resistance in range of 1 kΩ. This was checked for several hours to record any further fluctuations in the value. The sensor was then exposed to ammonia gas (air mixture) in range of 100-50 ppm, using a glass syringe and connecting silicon pipe.

Changes in resistance were observed with each pulse where a swift response time of few seconds was observed followed by a prolonged recovery. The ammonia pulses were repeated twice under similar conditions and the similarity in response was recorded.

Eventually, tests were done for lower ppm concentration and results were analyzed. Similar experiments were also performed with ammonia gas in nitrogen medium and changes following same pattern were observed.

### Example-5

Measured amount of H₂O₂ (5-10 ml) was added to 2-5 g of antimony doped tin oxide followed by drop-wise addition of water. The solution was allowed to undergo mixing for 2-4 hours to get a mixture. Then, 2-4 ml of pyrrole solution was added to 20 ml of D.I. water and then added to the mixture as prepared above. The obtained solution was then allowed to undergo prolonged heating at 120° C. for 5 hours. The solution was then centrifuged and washed with ethanol, dried at 80° C. for 3 hours and then drop-casted on the flat substrate.

The sensor was cured at 60° C. for 7 hours and connected to a multimeter for resistance measurements. The sensor was placed in a freezer. After a period of nearly 15-30 minutes, the sensor exhibited a stable base resistance in range of 1 W. This was checked for several hours to record any further fluctuations in the value. The sensor was then exposed to ammonia gas (air mixture) in range of 100-50 ppm, using a glass syringe and connecting silicon pipe.

Changes in resistance were observed with each pulse where a swift response time of few seconds was observed followed by a prolonged recovery. The ammonia pulses were repeated twice under similar conditions and the similarity in response was recorded.

Eventually, tests were done for lower ppm concentration and results were analyzed. Similar experiments were also performed with ammonia gas in nitrogen medium and changes following same pattern were observed.

During the experiments, the temperature of the freezer was varied from below room temperature to −30° C., changes in presence of ammonia individually recorded at intervals of 2° C. For much lower temperature of operation, dry ice (at −78° C.) was used as cooling agent and responses were recorded. Responses were also recorded in the high temperature range till 100° C. In the entire range of 100° C. to −78° C. the materials showed ammonia sensing property. However stable sensing response has been observed in the range of room temperature to −30° C.

### Advantages of the Invention

The antimony doped tin oxide-polymer nano composites provides many technical advantages. The main advantages of the present invention are presented hereinbelow:
i. Stable and reproducible very low temperature (below room temperature to −30° C.) in-situ and ex-situ sensing of low ppm ammonia gas.
ii. Below room temperature to −30° C. ammonia sensing by a novel but simple composition of antimony doped tin oxide-poly pyrrole nano composite.
iii. Hydrogen Peroxide (H₂O₂) has been used as a sole agent for both in-situ oxidative polymerization of pyrrole as well as for polypyrrole loading on antimony doped tin oxide during synthesis.
iv. The standard operational temperature for a metal oxide based gas/vapor sensor has been expanded in the very low temperature zone (below room temperature to −30° C.).

1. A composition for very low temperature sensing of low concentration ammonia gas, wherein, the composition comprises an antimony doped tin oxide-polypyrrole nano-composite, and wherein the composition sense 5-100 ppm of ammonia gas at a temperature range from below room temperature to −30° C.

2. The composition as claimed in claim 1, wherein the antimony doped tin oxide comprises 2-9 atomic % of antimony.

3. The composition as claimed in claim 1, wherein the antimony doped tin oxide-polypyrrole nano-composite comprises 1-2.5 wt. % of polypyrrole.

4. The composition as claimed in claim 1, wherein a pyrrole solution (1 M-2.5 M in water) is polymerized in-situ while loading on an antimony doped tin oxide.

5. The composition as claimed in claim 1, wherein a particle size of the antimony doped tin oxide is in a nano particle size range 10 nm to 30 nm, wherein, the nano particle size increases a surface to volume ratio and improves overall sensing response.

6. The composition as claimed in claim 1, wherein the composition shows a stable base resistance at temperature below room temperature to −30° C.

7. A method for preparing a composition for very low temperature sensing of low concentration ammonia gas, wherein, the method comprises steps of:
polymer loading, which uses hydrogen peroxide (H₂O₂) for simultaneous oxidative polymerization of pyrrole; and
loading of in-situ synthesized polypyrrole on the antimony (2-9 atomic %) doped tin oxide matrix.

8. The method as claimed in claim 7, wherein, the composition is a metal oxide-polymer nano-composite which provides a sensing response to 5-100 ppm ammonia gas at low temperature ranging below room temperature to −30° C.

9. The method as claimed in claim 7, wherein the composition provides a selective, stable and reproducible sensing response to a low ppm ammonia gas at a low temperature range, the composition provides sensing response to 5-100 ppm of ammonia gas at the low temperature range from below room temperature to −30° C.

10. A method for sensing ammonia gas under in-situ conditions and at a low temperature of from −30° C. to below room temperature, wherein the method comprises embedding the composition as claimed in claim 1 in a zone where sensing of ammonia gas is required.
